(19) United States
(12) Patent Application Publication
(10) Pub. No.: US 2005/0207997 A1
(43) Pub. Date: Sep. 22, 2005

(54) Dual component dentifrices and methods of whitening using the same

(75) Inventors: Nagaraj Dixit, Plainsboro, NJ (US); Diego A. Hoic, Jersey City, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/083,505

(22) Filed: Mar. 18, 2005

Related U.S. Application Data
(60) Provisional application No. 60/554,149, filed on Mar. 18, 2004.

(51) Int. Cl. A61K 7/16; A61K 7/18; A61K 7/20

(52) U.S. Cl. 424/52; 424/53

(57) ABSTRACT

An oral care composition comprises two components. A first such component comprises a first orally acceptable vehicle, a whitening agent and a fluoride salt providing fluoride ion in a total amount of at least about 1,200 ppm of the gel component. A second such component includes a second orally acceptable vehicle and a component that is incompatible with the whitening agent. The second component can comprise a clay-based thickening agent.

### CROSS REFERENCE TO RELATED U.S. APPLICATIONS

This application claims the benefit of U.S. Provisional Application No. 60/554,149 filed Mar. 18, 2004, the contents of which are incorporated herein by reference.

### BACKGROUND OF THE INVENTION

Dentifrices that not only clean oral surfaces but additionally provide a whitening effect on teeth have become highly popular. Whitening agents such as peroxy compounds are often ingredients of the dentifrices, but these compounds present formulation challenges in the preparation of products. For example, many common dentifrice ingredients, including certain abrasives, antibacterial agents and anticalculus agents, may contribute to the degradation of peroxy compounds, leading to unacceptable storage stability and/or short shelf life of a composition having these components.

Various types of dual component whitening dentifrices containing a peroxy compound and an ingredient incompatible with the peroxide, each physically segregated until dispensed for use, are described in the art. However, it would be desirable to provide further dual component dentifrice compositions having a whitening agent-containing gel component that exhibit acceptable Theological and storage stability properties. Moreover, as the components of dual tube dentifrices are often co-extruded from a dual tube dispenser, it may be desirable that each composition has a similar flow rate.

### BRIEF SUMMARY OF THE INVENTION

The invention described herein is directed to dual component dentifrices and methods of whitening dental surfaces using these dentifrices. Specifically, the invention provides an oral care composition that includes a (1) first component comprising a first orally acceptable vehicle; a whitening agent; and a fluoride salt that provides fluoride ion in an amount of at least about 1,200 ppm; and (2) a second component comprising a second orally acceptable vehicle and a compound that is incompatible with the whitening agent. The first component and the second component are separated from one another until applied to a dental surface.

Also described is a dispensing container for use with the dentifrice of the invention. The container has a collapsible sidewall, a septum defining a first chamber and a second chamber within the container, and a neck portion defining an openable and reclosable outlet. Each of the chambers within the container terminates in the outlet. The first chamber contains a first component that contains a first orally acceptable vehicle, a whitening agent and a fluoride salt providing fluoride ion in a total amount of at least about 1,200 ppm of the first component and the second chamber contains a second component comprising a second orally acceptable vehicle and a compound that is incompatible with the whitening agent.

Provided is a method for whitening a dental surface that includes contacting a first component and a second component to the dental surface substantially simultaneously using an applicator with agitation. The first component includes a first orally acceptable vehicle; a whitening agent; and a fluoride salt that provides fluoride ion in an amount of at least about 1,200 ppm; and the second component comprises a second orally acceptable vehicle and a compound that is incompatible the whitening agent.

### DETAILED DESCRIPTION OF THE INVENTION

It has been discovered that a fluoride salt present in greater concentrations than are utilized in conventional dual component preparations in a whitening agent-containing gel component of a dual-component dentifrice, results in increase in the viscosity of the gel component.

A “dental surface” as used herein is a surface of a natural tooth or a hard surface of artificial dentition including a crown, cap, filling, bridge, dental implant and the like. An “orally acceptable” compound, composition, or vehicle is one that is not harmful to a mammal in amounts disclosed herein when retained in the mouth, without swallowing, for a period sufficient to permit application to a dental surface as required. Preferably, the compound, composition or vehicle is not harmful to the mammal if swallowed.

When amounts of ingredients are recited herein as concentrations, e.g., in percent (%) or parts per million (ppm), these are expressed as concentrations in the first or second component of the composition, not in the composition as a whole unless otherwise indicated.

Classification herein of an ingredient as an active or a carrier ingredient is made for clarity and convenience, and no inference should be drawn that a particular ingredient necessarily functions solely in the composition in accordance with its classification herein. Furthermore, a particular ingredient can serve a plurality of functions, thus disclosure of an ingredient herein as exemplifying one functional class does not exclude the possibility that it can also exemplify another functional class.

As used herein a “safe and effective” amount is an amount sufficient to provide a desired benefit, for example a therapeutic or prophylactic effect, when the composition is used repeatedly, without undue side effects such as toxicity, irritation or allergic reaction, commensurate with a reasonable benefit/risk ratio. Such a safe and effective amount will usually, but not necessarily, fall within ranges approved by appropriate regulatory agencies. A safe and effective amount in a specific case depends on many factors, including the particular benefit desired or condition being treated or sought to be prevented, the particular subject using, or being administered, the composition, the frequency and duration of use, etc.

As indicated above, an oral care composition of the invention comprises a first component and a second component. Each component is of a viscosity suitable for use as a non-powder dentifrice, although the precise nature and form of the component will vary. It is preferred that each or both of the components is in a semi-solid form. A semi-solid component is one that can be induced to flow from an outlet of a container having a collapsible sidewall on application of pressure to the sidewall, and that stands up, i.e., does not substantially deform or flow when deposited on an applicator such as a toothbrush. Semi-solid form which the components of the invention may take include pastes, gels, and high viscosity liquids, but not powder dentifrices. Preferably, the first component is a gel and the second component is a paste.

The first component of the oral care composition of the invention contains a whitening agent. Any orally acceptable whitening agent or combination of agents known or to be developed in the art may be used. Suitable whitening agents include, without limitation, peroxy compounds, chlorine dioxide, and chlorites and hypochlorites. Preferred chlorites and hypochlorites include those of alkali and alkaline earth metals such as lithium, potassium, sodium, magnesium, calcium and barium. Alternatively or in addition, one or more peroxy compounds can be used. Suitable peroxy compounds include any orally acceptable compound(s) that delivers a perhydroxy (OOH⁻) ions, hydrogen peroxide, peroxides of alkali and alkaline earth metals, organic peroxy compounds, and peroxy acids and salts thereof. Peroxy compounds for use in the composition of the invention can optionally be present in a form of a polymer-peroxide complex, for example, a polyvinylpyrrolidone-hydrogen peroxide complex.

Peroxides of alkali and alkaline earth metals include lithium peroxide, potassium peroxide, sodium peroxide, magnesium peroxide, calcium peroxide, and barium peroxide.

Organic peroxy compounds include, for example, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate and the like.

Peroxy acids and their salts include organic peroxy acids such as alkyl peroxy acids and monoperoxyphthalate, as well as inorganic peroxy acid salts including persulfate, dipersulfate, percarbonate, perphosphate, perborate and persilicate salts of alkali and alkaline earth metals such as lithium, potassium, sodium, magnesium, calcium and barium. Another useful peroxy compound is sodium pyrophosphate peroxyhydrate.

The whitening agent is present in the first component in an amount effective to result in whitening of a dental surface when applied to that surface over a selected treatment regime. Accordingly, the amount of whitening compounds present will necessarily depending on the desired duration of the treatment regime and/or the degree of whitening desired.

If using peroxy compounds, they may preferably be present in a hydrogen peroxide equivalent amount of about 0.1% to about 10%, for example about 1% to about 5%, by weight of the first component.

The first component of the oral composition a fluoride salt that provides to the composition a source of fluoride ions. Any orally acceptable fluoride salt or combination of salts is used, including without limitation alkali metal fluorides (e.g., potassium, sodium), ammonium fluoride, stannous and indium fluorides and the like.

One or more fluoride salts are present in the first component in an amount providing at least about 1,200 ppm of fluoride ions. Preferably, the fluoride salt(s) are present in an amount that provides about 1,200 to about 20,000 ppm, about 1,200 to about 5,000 ppm, or about 1,200 to about 2,500 ppm of fluoride ions. In one embodiment, the one or more fluoride salts in the gel component provide a total of at least about 1,600 ppm, for example about 1,600 to about 20,000 ppm, about 1,600 to about 5,000 ppm, or about 1,600 to about 2,500 ppm, fluoride ions. In another embodiment, the one or more fluoride salts in the gel component provide a total of at least about 2,000 ppm, for example about 2,000 to about 20,000 ppm, about 2,000 to about 5,000 ppm, or about 2,000 to about 2,500 ppm, fluoride ions.

Where sodium fluoride is the sole fluoride salt present in the composition of the invention, illustratively, an amount of at least about 0.26%, for example about 0.26% to about 4.4%, about 0.35% to about 1.1% or about 0.44% to about 0.55%, sodium fluoride by weight can be present in the gel component.

If desired, a source of fluoride ions such as a fluoride or monofluorophosphate salt can also be present in the second component. In such a case, the amount of fluoride sources in the composition as a whole can be sufficient to provide a total of up to about 20,000 ppm, for example about 800 to about 20,000 ppm, fluoride ions in the composition.

A second component is included in the oral care composition of the invention. The second component includes compound that is incompatible with the whitening agent. An incompatible compound is one that, upon exposure to the whitening agent undergoes a chemical, physical or a combination of physical and chemical modification such that the desired function of the compound or agent is substantially impaired. Examples of such modifications include oxidation of the compound or agent. Alternatively or additionally, a compound or agent is “incompatible” with a selected whitening agent if, upon exposure to the compound or agent, the whitening agent undergoes a chemical, physical or physio-chemical modification, for example, the evolution of oxygen from a peroxy compound.

Incompatible compounds suitable for inclusion in the second component include without limitation, siliceous abrasives, aluminous abrasives, antibacterial agents, anticalculus agents, and peroxide activators.

Optionally, the second component may include at least one peroxide activator. Any orally acceptable peroxide activator can be used, including without limitation iron ion-implanted clays and manganese coordination complex compounds such as those disclosed in U.S. Pat. No. 5,648,064, incorporated herein by reference. Preferred may be manganese gluconate.

Preferably the manganese coordination complex compound(s) may be present in a total amount of about 0.005% to about 3%, for example about 0.01% to about 0.5%, by weight of the second component.

Where the second component is a paste, for example a paste comprising a siliceous and/or aluminous abrasive, this paste component in one embodiment further comprises a clay-based thickening agent. Any orally acceptable clay-based thickening agent can be used, including such agents comprising natural, modified and/or synthetic clays. Illustratively, thickening agents comprising at least one clay of the smectite class, including beidellite, bentonite, hectorite, montmorillonite, saponite and stevensite, and synthetic counterparts such as colloidal magnesium aluminum silicate and LAPONITE® are useful. Hydrophobically modified clays such as hydrophobically modified bentonite are also useful. One or more clay-based thickening agents are optionally present in the second component in a thickening or viscosity increasing effective total amount, typically about 0.1% to about 2%, for example about 0.3% to about 1%, by weight of the second component.

The second component can optionally comprise a non-clay-based thickening agent, including an organic thickening agent such as those disclosed hereinabove and/or a siliceous thickening agent such as colloidal silica.

Each of the first and second components includes a vehicle or carrier that “carries” the components ingredients. Carriers to be included in the first or second component should be selected for compatibility with the other ingredients of that component. Among useful carriers are diluents, bicarbonate salts, pH modifying agents, surfactants, foam modulators, activating agents for particular oral care actives including peroxide activators, stabilizing agents for particular oral care actives including peroxide stabilizers, thickening agents, viscosity modifiers, mouth feel modifying agents, humectants, sweeteners, flavorants and colorants. One carrier material, or more than one carrier material of the same or different classes, can optionally be present. As is understood to a person of skill in the art, the carrier or carriers selected will vary depending on the ingredients in the component and/or the desired form of the component. For example, in the form is a gel, the desired carrier may be water.

Each of the first and the second components may contain one or more additional additive or ingredients to facilitate oral or systemic health, as long as the selected additive(s) do not substantially impair or erode the functionality of the active ingredients in each component. Examples of these additives are provided below. Each component may contain one or more of the listed additives, and the additives in each may be the same or different.

For example, the second and/or first component may include at least one abrasive, useful for example as a cleaning and/or polishing agent. Any orally acceptable abrasive can be used, but type, fineness (particle size) and amount of abrasive should be selected so that tooth enamel is not excessively abraded in ordinary use of the composition. Suitable abrasives include without limitation silica, for example in the form of silica gel, hydrated silica, pyrogenic silica or precipitated silica, alumina, for example in the form of hydrated alumina or calcined alumina, aluminum silicate, bentonite, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. One or more abrasives are optionally present in the second component in any amount sufficient to effect an abrasive action. Preferred amounts may be about 5% to about 70%, for example about 10% to about 50% or about 15% to about 30% by weight. The average particle size of an abrasive, if present, may be preferably about 0.1 to about 30 μm, about 1 to about 20 μm or about 5 to about 15 μm.

Among the listed abrasives, siliceous and/or aluminous abrasives including silica, hydrated silica, pyrogenic silica, silica gels and precipitates, alumina, hydrated alumina, calcined alumina, aluminum silicate and bentonite, when used in abrasive effective amounts, are typically incompatible with peroxy compounds, in large measure because of transition metal impurities that can be present in mineral products such as these. Such incompatible abrasives should therefore be formulated only in the second or paste component of the composition. Abrasives such as insoluble phosphates that are not incompatible with peroxy compounds can, if desired, be formulated in either or both of the first and second components. One or more siliceous and/or aluminous abrasives, for example hydrated silica, may preferably be present in a total amount of about 15% to about 30% by weight of the second component.

The second component can optionally include a first abrasive selected primarily for high cleaning efficacy and a second abrasive selected primarily for polishing efficacy and/or enhanced mouth feel. Such first and second abrasives are herein termed “high-cleaning” and “prophy” abrasives respectively. For example, a high-cleaning silica and a prophy silica can be included, each illustratively in a total amount of about 5% to about 15% by weight of the second component.

One or more antimicrobial or antibacterial agents may be included in the first or second components. Any orally acceptable antimicrobial agent can be used, including without limitation triclosan (5-chloro-2-(2,4-dichlorophenoxy)phenol), 2,2′-dihydroxy-5,5′-dibromodiphenyl ether, 8-hydroxyquinoline and salts thereof, copper (II) compounds such as copper (II) chloride, fluoride, sulfate and hydroxide, zinc ion sources such as zinc citrate, zinc sulfate, zinc glycinate and sodium zinc citrate, phthalic acid and salts thereof such as magnesium monopotassium phthalate, hexetidine, octenidine, sanguinarine, benzalkonium chloride, salicylanilide, domiphen bromide, alkylpyridinium chlorides such as cetylpyridinium chloride (CPC) (including combinations of CPC with zinc and/or enzymes), tetradecylpyridinium chloride and N-tetradecyl4-ethylpyridinium chloride, octenidine, iodine, sulfonamides, bisbiguanides such as alexidine, chlorhexidine and chlorhexidine digluconate, phenolics, piperidino derivatives such as delmopinol and octapinol, magnolia extracts, grapeseed extract, phenol, thymol, eugenol, menthol, geraniol, carvacrol, citral, eucalyptol, catechol, 4-allylcatechol, hexyl resorcinol, halogenated bisphenolics such as 2,2′-methylene bis(4-chloro-6-bromophenol), methyl salicylate, antibiotics such as augmentin, amoxicillin, tetracycline, doxycycline, minocycline, metronidazole, neomycin, kanamycin and clindamycin, and the like. Other suitable antibacterial agents include those listed in U.S. Pat. No. 5,776,435 to Gaffar et al. incorporated herein by reference.

Among antimicrobial agents, some nonionic agents such as halogenated diphenylethers (e.g., triclosan and 2,2′-dihydroxy-5,5′-dibromodiphenyl ether) and phenolic compounds may be incompatible with peroxy compounds and should therefore be formulated only in the second component of the composition.

These agents may be present in any antimicrobially effective amount; however, it may be preferred that the component(s) contain the agent in an amount of about 0.05% to about 3%, for example about 0.1% to about 1% by weight.

Anticalculus agents that may be included in either of the components, if desired, although care should be taken not to include those that are incompatible with peroxy compounds if incorporating the agent into the first component. With this proviso, any orally acceptable anticalculus agent can be used, including without limitation phosphates and polyphosphates (for example pyrophosphates), polyaminopropane-sulfonic acid (AMPS), zinc citrate trihydrate, polypeptides such as polyaspartic and polyglutamic acids, polyolefin sulfonates, polyolefm phosphates, diphosphonates such as azacycloalkane-2,2-diphosphonates (e.g., azacycloheptane-2,2-diphosphonic acid), N-methyl azacyclopentane-2,3-diphosphonic acid, ethane-l-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate, tetrapolyphosphate, mono-, di-, tri- and tetrasodium pyrophosphates, disodium dihydrogen pyrophosphate, sodium trimetaphosphate, sodium hexametaphosphate and the like, wherein sodium can optionally be replaced by potassium or ammonium. Other useful anticalculus agents include polycarboxylate polymers and polyvinyl methyl ether/maleic anhydride (PVME/MA) copolymers, such as those available under the commercial mark GANTREZ® from ISP, Wayne, N.J., United States of America.

Preferably, one or more anticalculus agents are optionally present in the first and/or second component in an anticalculus effective total amount, typically about 0.01% to about 50%, for example about 0.05% to about 25% or about 0.1% to about 15% by weight.

If a PVME/MA copolymer(s) are used, they may be present in a total amount of about 0.3% to about 3% by weight of the component(s), optionally together with one or more polyphosphate salts, e.g., tetrasodium pyrophosphate, tetrapotassium pyrophosphate, sodium tripolyphosphate and/or potassium tripolyphosphate, in a total amount of about 1% to about 15% by weight.

Additionally or alternatively, one or both of the first and second components may include at least one stannous ion source. Any orally acceptable stannous ion source can be used, including without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5% by weight of the composition as a whole.

Antioxidants may be present in the one or both of the components of the invention. Any orally acceptable antioxidant can be used, including without limitation butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin and the like. One or more antioxidants are optionally present in an antioxidant effective total amount. In a particular embodiment at least one of BHA and BHT is present in the gel component in a total amount of about 0.01% to about 0.1% by weight.

The composition may comprise, in one or both of the first and second components, a sialagogue (saliva stimulating agent), useful for example in amelioration of dry mouth. Any orally acceptable sialagogue can be used, including without limitation food acids such as citric, lactic, malic, succinic, ascorbic, adipic, fumaric and tartaric acids. One or more sialagogues are optionally present in the composition in a saliva stimulating effective total amount.

A breath freshening agent may be included in the components. Any orally acceptable breath freshening agent can be used, including without limitation zinc salts such as zinc gluconate, zinc citrate and zinc chlorite, α-ionone and the like. One or more breath freshening agents are optionally present in the composition in a breath freshening effective total amount.

The composition may comprise in one or both of the first and dentifrice components, an antiplaque, including plaque disrupting, agent. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and chelating agents such as citric and tartaric acids and alkali metal salts thereof. One or more antiplaque agents are optionally present in the composition in an antiplaque effective total amount.

The composition may comprise in one or both of the first and second components, at least one anti-inflammatory agent. Any orally acceptable anti-inflammatory agent can be used, including without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in the composition in an anti-inflammatory effective amount.

In a still further embodiment the composition comprises, in one or both of the first and second components, at least one desensitizing agent. Potassium salts such as potassium citrate, potassium tartrate, potassium chloride, potassium sulfate and potassium nitrate are illustratively useful in this regard, as is sodium nitrate. Alternatively or in addition a local or systemic analgesic such as aspirin, codeine, acetaminophen, sodium salicylate or triethanolamine salicylate can be used. One or more densitizing agents and/or analgesics are optionally present in the composition in a desensitizing and/or analgesic effective amount.

The composition may contain, in one or both of the first and second components, at least one nutrient. Suitable nutrients include vitamins, minerals and amino acids.

The composition may contain, in one or both of the first and second components, at least one bicarbonate salt, useful for example to impart a perceived “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate and the like. One or more bicarbonate salts are optionally present in a total amount of 0.1% to about 50%, for example about 1% to about 20% by weight of the composition as a whole.

In a still further embodiment the composition comprises, in one or both of the first and second components, at least one pH modifying agent. Such agents include acidifying agents to lower pH, basifying agents to raise pH and buffering agents to control pH within a desired range. For example, one or more compounds selected from acidifying, basifying and buffering agents can be included to provide a pH of about 2 to about 10, or in various illustrative embodiments about 2 to about 8, about 3 to about 9, about 4 to about 8, about 5 to about 7, about 6 to about 10, about 7 to about 9, etc. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like. One or more pH modifying agents are optionally present in a total amount effective to maintain each component of the composition in an orally acceptable pH range.

In a still further embodiment the composition comprises, in one or both of the first and second components, at least one surfactant, useful for example to compatibilize other ingredients and thereby provide enhanced stability, to help in cleaning the dental surface through detergency, and to provide foam upon agitation, e.g., during brushing. Any orally acceptable surfactant, including cationic, anionic, nonionic and amphoteric types, can be used.

Suitable cationic surfactants include without limitation quaternary ammonium compounds with a C₈₋₂₀ aliphatic chain such as lauryl trimethylammonium chloride, cetyl pyridinium chloride, cetyl pyridinium fluoride, cetyl trimethylammonium bromide, diisobutylphenoxyethyl-dimethylbenzylammonium chloride, cocoalkyltrimethylammonium nitrite and the like. Cationic compounds that can stain teeth, for example chlorhexidine, can be considered for use herein, bearing this disadvantage in mind.

Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium lauryl sulfoacetate, sodium laureth carboxylate, sodium dodecyl benzenesulfonate and sodium and potassium salts of lauroyl sarcosinate, myristoyl sarcosinate, palmitoyl sarcosinate, stearoyl sarcosinate and oleoyl sarcosinate.

Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like.

Suitable amphoteric surfactants include without limitation derivatives of C₈₋₂₀ aliphatic secondary and tertiary amines having an anionic group such as carboxylate, sulfate, sulfonate, phosphate or phosphonate. Examples include cocoamidopropyl betaine and lauramidopropyl betaine.

One or more surfactants are optionally present in a total amount of about 0.01% to about 10%, for example about 0.05% to about 5% or about 0.1% to about 2% by weight of the composition as a whole.

In a still further embodiment the composition comprises, in one or both of the first and second components, at least one foam modulator, useful for example to increase amount, thickness or stability of foam generated by the composition upon agitation, e.g., brushing. Any orally acceptable foam modulator can be used, including without limitation polyethylene glycols (PEGs), also known as polyoxyethylenes. High molecular weight PEGs are suitable, including those having an average molecular weight of about 200,000 to about 7,000,000, for example about 500,000 to about 5,000,000 or about 1,000,000 to about 2,500,000. One or more PEGs are optionally present in a total amount of about 0.1% to about 10%, for example about 0.2% to about 5% or about 0.25% to about 2% by weight of the composition as a whole.

In a still further embodiment the composition comprises, in one or both of the first and second components, at least one humectant, useful for example to prevent hardening of the composition or a component thereof upon exposure to air, and/or to enhance mouth feel. Any orally acceptable humectant can be used, including without limitation polyhydric alcohols such as propylene glycol, butylene glycol, glycerin, sorbitol, xylitol or low molecular weight PEGs. Most humectants also function as sweeteners. One or more humectants are optionally present in a total amount of about 1% to about 80%, for example about 5% to about 65% or about 10% to about 50% by weight of the composition as a whole.

In a particular embodiment, the first component may be glycerin in an amount of about 10% to about 60% by weight, optionally together with a low molecular weight PEG such as PEG 600 in an amount of about 2% to about 20% by weight of the gel component.

Optionally, the second component is a paste component comprising sorbitol in an amount of about 10% to about 50%, optionally together with glycerin in an amount of about 5% to about 25% by weight of the paste component.

In a still further embodiment the composition comprises, in one or both of the first and second components, at least one sweetener, useful for example to enhance taste of the composition. Any orally acceptable natural or artificial, nutritive or non-nutritive sweetener can be used, including without limitation dextrose, polydextrose, sucrose, maltose, dextrin, dried invert sugar, lactose, mannose, xylose, ribose, fructose, galactose, corn syrup (including high fructose corn syrup and corn syrup solids), partially hydrolyzed starch, hydrogenated starch hydrolysate, sorbitol, mannitol, xylitol, maltitol, isomalt, sucralose, aspartame, acesulfame, neotame, D-tryptophan, saccharin and salts thereof (e.g., sodium saccharin), thaumatin, dihydrochalcones, dipeptide-based intense sweeteners, cyclamates (e.g., sodium cyclamate) and the like. One or more sweeteners are optionally present in a total amount depending strongly on the particular sweetener(s) selected, but typically about 0.005% to about 5% by weight of the composition as a whole.

In a particular embodiment, each of the first and second components comprises sodium saccharin in an amount of about 0.1% to. about 1% by weight.

In a still further embodiment the composition comprises, in one or both of the first and second components, at least one flavorant, useful for example to enhance taste of the composition. Any orally acceptable natural or synthetic flavorant can be used, such as oils, aldehydes, esters, alcohols and the like, and mixtures, including multi-component mixtures, thereof. Flavorants include without limitation vanillin, sage, marjoram, parsley oil, spearmint oil, cinnamon oil, oil of wintergreen (methyl salicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, a-irisone, propenyl guaiethol, thymol, linalool, benzaldehyde, cinnamaldehyde, N-ethyl-p-menthan-3-carboxamide, N,2,3-trimethyl-2-isopropylbutanamide, 3-1-menthoxypropane-1,2-diol, cinnamaldehyde glycerol acetal (CGA), menthone glycerol acetal (MGA), capsicum, benzyl nicotinate and the like. One or more flavorants are optionally present in a total amount of about 0.01% to about 5%, for example about 0.1% to about 2.5% by weight of the composition as a whole.

In a still further embodiment the composition comprises, in one or both of the first and second components, at least one colorant. Colorants herein include pigments, dyes, lakes and agents imparting a particular luster or reflectivity such as pearling agents. A colorant can serve a number of functions, including for example to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of about 0.001% to about 20%, for example about 0.01% to about 10% or about 0.1% to about 5% by weight of the composition as a whole.

In a particular embodiment, the first and second components have contrasting colors to provide a striped effect upon extrusion from an outlet of a dual-chamber container onto an applicator such as a toothbrush. For example, the gel component can contain a blue colorant and the paste component can contain titanium dioxide to appear white.

Preferably, the first component and the second component may each independently be a gel prepared by mixing the ingredients in any suitable mixing device.

Where the second component is a paste component, it can be prepared by the following general procedure. Water and thickening agent(s), typically together with humectant(s) and sweetening agent(s), are mixed in a suitable mixing device until a homogeneous gel phase is obtained. Into the gel phase other ingredients, such as pigment(s) and fluoride ion source(s), can be added with further mixing until homogeneous. Thereafter, abrasive(s) and/or other desired ingredients such as anticalculus agent(s), antibacterial agent(s), flavorant(s) and surfactant(s) are added and the resulting mixture is mixed at high speed, optionally under vacuum of about 20 to about 100 mm Hg, to provide a homogeneous extrudable paste.

Relative amounts of the first and second components are not narrowly critical. In one embodiment the first and second components, for example a gel and paste component respectively, are present in a weight ratio of about 1:3 to about 3:1, for example about 1:2 to about 2:1, for example about 1:1.5 to about 1.5:1. In a particular embodiment the amounts of the first and second components are substantially equal. For example, an illustrative composition comprises about 40% to about 60% by weight of a gel component and about 60% to about 40% by weight of a paste component as described herein.

The dual-component composition of the invention can be packaged in a suitable dispensing container in which the first and second components are maintained in physical isolation from one another and from which they can be dispensed synchronously. Such containers are known in the art. An example of such a container is a dual-chamber dentifrice tube comprising a collapsible sidewall, a septum defining a first chamber and a second chamber within the container, and a neck portion defining an openable and reclosable outlet, wherein both of the chambers terminate in the outlet. Illustratively, the container can be substantially as disclosed in U.S. Pat. No. 4,487,757 to Kiozpeoplou, incorporated herein by reference.

As an alternative, the container can be substantially as disclosed in U.S. Pat. No. 4,687,663 to Schaeffer, incorporated herein by reference.

As a further alternative, the container can be substantially as disclosed in U.S. Pat. No. 5,927,550 to Mack et al., incorporated herein by reference.

As a still further alternative, the container can be a dual chamber pump, for example substantially as disclosed in U.S. Pat. No. 6,230,935 to Mack et al., incorporated herein by reference.

A method for whitening a dental surface comprises extruding from an outlet of a dual-chamber dispensing container a composition as provided herein onto an applicator, and thereafter applying the composition to the dental surface with agitation of the applicator. Typically the applicator is a toothbrush and agitation is effected by brushing the dental surface with the toothbrush after dispensing a suitable amount of the composition onto the toothbrush. Wetting the applicator before, during and/or after extruding the composition onto the applicator can be helpful in assuring effective application of the composition to the dental surface. The dental surface can be rinsed with water after brushing. Brushing for at least about 30 seconds, or at least about one minute, or at least about two minutes can be desirable to achieve the desired whitening effect. The method can be repeated as frequently and as many times as desired or necessary.

The dental surface to be whitened by the method of the invention can be in a human or nonhuman subject, for example a nonhuman mammalian subject such as a companion animal, for example a dog or cat. In one embodiment the dental surface is a surface of one or more natural teeth, but the method is also applicable to a surface of artificial dentition, for example a crown, a cap, a filling, a bridge or a dental implant.

The invention can further be understood by reference to the following nonlimiting examples.

### EXAMPLES

### Example 1

Gel formulations designated A to E were prepared as a first semi-solid dentifrice component of a dual-component oral care composition, following the procedure generally described for a first component above. Composition of each of formulations A-E is shown in Table 1. All ingredients except sodium fluoride, LAPONITE® D and water were present in identical amounts in all five formulations. Sodium fluoride in a concentration of 0.243% provides about 1,100 ppm fluoride ion, and in a concentration of 0.486% provides about 2,200 ppm fluoride ion.

TABLE 1
Composition of gel formulations A-E

| | Weight % | | | | |
|---|---|---|---|---|---|
| Ingredient | A | B | C | D | E |
| hydrogen peroxide, | 5.71 | 5.71 | 5.71 | 5.71 | 5.71 |
| 35% in water | | | | | |
| sodium fluoride | 0.486 | 0.486 | 0.486 | 0.486 | 0.243 |
| CARBOPOL ® | 2.10 | 2.10 | 2.10 | 2.10 | 2.10 |
| 974 (carbomer) | | | | | |
| Xanthan | 0.40 | 0.40 | 0.40 | 0.40 | 0.40 |
| LAPONITE ® D | 0.10 | 0.05 | 0.02 | 0 | 0.10 |
| (clay-based | | | | | |
| thickening agent) | | | | | |
| silica thickening agent | 0.30 | 0.30 | 0.30 | 0.30 | 0.30 |
| Glycerin | 40.00 | 40.00 | 40.00 | 40.00 | 40.00 |
| PEG 600 | 10.00 | 10.00 | 10.00 | 10.00 | 10.00 |
| sodium saccharin | 0.25 | 0.25 | 0.25 | 0.25 | 0.25 |
| Flavorant | 1.15 | 1.15 | 1.15 | 1.15 | 1.15 |
| Colorant | 0.28 | 0.28 | 0.28 | 0.28 | 0.28 |
| BHT | 0.03 | 0.03 | 0.03 | 0.03 | 0.03 |
| phosphoric acid | 0.10 | 0.10 | 0.10 | 0.10 | 0.10 |
| Water | q.s. | q.s. | q.s. | q.s. | q.s. |

Average viscosity of each of gel formulations A-E was measured two to four days after preparation, using a Brookfield viscometer with an “E” spindle. Results are shown in Table 2.

TABLE 2
Average viscosity of gel formulations A-E

| | Fluoride | | |
|---|---|---|---|
| Formulation | (ppm) | LAPONITE ™ D (%) | Viscosity (×10,000 cP) |
| A | 2,200 | 0.10 | 39 |
| B | 2,200 | 0.05 | 34 |
| C | 2,200 | 0.02 | 32 |
| D | 2,200 | 0 | 29 |
| E | 1,100 | 0.10 | 28 |

It can be seen from Table 2 that, in presence of 0.1% LAPONITE® clay, increasing fluoride concentration from 1,100 to 2,200 ppm (formulation A) resulted in an approximately 30% increase in viscosity of the gel formulation by comparison with formulation E. Removal of the clay while increasing fluoride concentration from 1,100 to 2,200 ppm (formulation D) resulted in viscosity similar to that of the comparative formulation E.

These findings indicate a new and alternative approach to viscosity control for a whitening gel component of a dual-component dentifrice product.

### Example 2

Paste formulations designated 1 to 4, as shown in Table 3, were prepared as a second semi-solid dentifrice component of a dual-component oral care composition in accordance with the procedure generally described for a paste component above. All formulations contained LAPONITE® D in an amount of 0.75% by weight of the paste formulation. All ingredients except CMC sodium, sorbitol, ZEODENT® 165 and water were present in identical amounts in all four formulations.

TABLE 3
Composition of paste formulations 1-4

| | Weight % | | | |
|---|---|---|---|---|
| Ingredient | 1 | 2 | 3 | 4 |
| SYLODENT ® 783 (silica abrasive) | 11.00 | 11.00 | 11.00 | 11.00 |
| SYLODENT ® XWA 650 (silica abrasive) | 10.00 | 10.00 | 10.00 | 10.00 |
| LAPONITE ® (clay-based thickening agent) | 0.75 | 0.75 | 0.75 | 0.75 |
| CMC sodium | 0.95 | 0.90 | 0.90 | 0.85 |
| ZEODENT ® 165 (silica thickening agent) | 1.20 | 1.20 | 1.70 | 1.20 |
| sorbitol | 27.60 | 27.60 | 27.10 | 27.60 |
| glycerin | 12.00 | 12.00 | 12.00 | 12.00 |
| ι-carrageenan | 0.35 | 0.35 | 0.35 | 0.35 |
| PVM/MA, 13% in water | 7.69 | 7.69 | 7.69 | 7.69 |
| tetrasodium pyrophosphate | 1.00 | 1.00 | 1.00 | 1.00 |
| sodium tripolyphosphates | 7.00 | 7.00 | 7.00 | 7.00 |
| sodium lauryl sulfate, 29% in water | 7.33 | 7.33 | 7.33 | 7.33 |
| manganese gluconate | 0.05 | 0.05 | 0.05 | 0.05 |
| sodium saccharin | 0.55 | 0.55 | 0.55 | 0.55 |
| flavorant | 1.15 | 1.15 | 1.15 | 1.15 |
| titanium dioxide | 1.00 | 1.00 | 1.00 | 1.00 |
| sodium hydroxide, 50% in water | 2.00 | 2.00 | 2.00 | 2.00 |
| water | q.s. | q.s. | q.s. | q.s. |

Viscosity of each of paste formulations 1 to 4 was measured using a Brookfield viscometer with an “E” spindle, at five times ranging from three to twenty-eight days after preparation of the formulations. Results are shown in Table 4.

TABLE 4
Viscosity of paste formulations 1-4

| | Viscosity (×10,000 cP) | | | | |
|---|---|---|---|---|---|
| Formulation | 3 days | 7 days | 14 days | 21 days | 28 days |
| 1 | 32 | 34 | 34 | 35 | 35 |
| 2 | 33 | 34 | 34 | 32 | 35 |
| 3 | 32 | 33 | 33 | 36 | 35 |
| 4 | 25 | 26 | 26 | 27 | 28 |

It can be seen from Table 4 that paste formulations 1-4 exhibit little upward drift in viscosite after seven days from preparation.

### Example 3

Gel formulation F and paste formulation 5, as shown in Table 5, were prepared as first and second semi-solid components respectively of a dual-component whitening dentifrice in accordance with the procedures generally described above. The components were packaged in a 45.1:54.9 ratio by weight in a dual-chamber dentifrice tube.

TABLE 5
Composition of dual-component whitening dentifrice

| | Weight % | | |
|---|---|---|---|
| | | | whole |
| Ingredient | gel F | paste 5 | composition |
| hydrogen peroxide, 35% in water | 5.71 | | 2.575 |
| sodium fluoride | 0.54 | | 0.243 |
| Sylodent ® 783 (silica abrasive) | | 11.00 | 6.039 |
| Sylodentc ® XWA 650 (silica abrasive) | | 10.00 | 5.490 |
| LAPONITE ® D (clay-based thickening | | 0.75 | 0.412 |
| agent) | | | |
| CMC sodium | | 0.95 | 0.522 |
| CARBOPOL ® 974P (carbomer) | 2.10 | | 0.947 |
| Xanthan | 0.40 | | 0.180 |
| ZEODENT ® 115 (silica thickening | 0.30 | | 0.135 |
| agent) | | | |
| ZEODENT ® 165 (silica thickening | | 1.70 | 0.933 |
| agent) | | | |
| Sorbitol | | 27.50 | 15.098 |
| Glycerin | 40.00 | 12.00 | 24.628 |
| PEG 600 | 10.00 | | 4.510 |
| carrageenan LB9505 | | 0.35 | 0.192 |
| PVM/MA, 13% in water | | 7.69 | 4.222 |
| tetrasodium pyrophosphate | | 1.00 | 0.549 |
| sodium tripolyphosphates | | 7.00 | 3.843 |
| sodium lauryl sulfate, 29% in water | | 7.33 | 4.024 |
| manganese gluconate | | 0.05 | 0.027 |
| sodium saccharin | 0.25 | 0.55 | 0.415 |
| flavorant for gel | 1.15 | | 0.519 |
| peppermint flavor | | 1.15 | 0.631 |
| colorant (Blue #1, 12% in water) | 0.27 | | 0.122 |
| titanium dioxide | | 1.00 | 0.549 |
| BHT | 0.03 | | 0.014 |
| phosphoric acid, 85% in water | 0.10 | | 0.045 |
| sodium hydroxide, 50% in water | | 2.00 | 1.098 |
| Water | q.s. | q.s. | q.s. |

1. An oral care composition comprising:
a first component comprising a first orally acceptable vehicle; a whitening agent; and a fluoride salt that provides fluoride ion in an amount of at least about 1,200 ppm; and
a second component comprising a second orally acceptable vehicle and a compound that is incompatible with the whitening agent;
wherein the first component and the second component are separated until applied to a dental surface.

2. The composition of claim 1, wherein the whitening agent comprises a peroxy compound.

3. The composition of claim 1 wherein the whitening agent comprises a compound selected from the group consisting of hydrogen peroxide, an organic peroxy compound, and a peroxy acid.

4. The composition of claim 1 wherein the fluoride salt provides fluoride ion in a total amount of about 1,200 to about 20,000 ppm of the first component.

5. The composition of claim 1 wherein the fluoride salt provides fluoride ion in a total amount of about 1,600 to about 5,000 ppm of the first component.

6. The composition of claim 1 wherein the fluoride salt is selected from an alkali metal fluoride, an ammonium fluoride, a stannous fluoride, and mixtures thereof.

7. The composition of claim 1 wherein the fluoride salt is sodium fluoride.

8. The composition of claim 1 wherein the first component is substantially free of a clay-based thickening agent.

9. The composition of claim 1 wherein the second component comprises an abrasive selected from a siliceous abrasive and an aluminous abrasive.

10. The composition of claim 1 wherein the second component comprises an abrasive selected from silica, hydrated silica, alumina, hydrated alumina, calcined alumina, aluminum silicate, and bentonite.

11. The composition of claim 1 wherein the second component comprises precipitated amorphous hydrated silica in an amount of about 10% to about 50% by weight of the second component.

12. The composition of claim 1 wherein the second component comprises precipitated amorphous hydrated silica in an amount of about 15% to about 40% by weight of the second component.

13. The composition of claim 1 wherein the second component comprises a clay-based thickening agent.

14. The composition of claim 15 wherein the clay-based thickening agent is present in an amount of about 0.1% to about 2% by weight of the second component.

15. The composition of claim 15 wherein the clay-based thickening agent is present in an amount of about 0.3% to about 1% by weight of the second component.

16. An dispensing container having a collapsible sidewall, a septum defining a first chamber and a second chamber within the container, and a neck portion defining an openable and reclosable outlet, both of the chambers terminating in the outlet; wherein the first chamber contains a first component comprising a first orally acceptable vehicle, a whitening agent and a fluoride salt providing fluoride ion in a total amount of at least about 1,200 ppm of the first component and the second chamber contains a second component comprising a second orally acceptable vehicle and a compound that is incompatible with the whitening agent.

17. The container of claim 18 wherein the second component comprises a clay-based thickening agent and an abrasive selected from a siliceous abrasive and an aluminous abrasive.

18. A method for whitening a dental surface comprising contacting a first component and a second component to the dental surface substantially simultaneously using an applicator with agitation, wherein the first component comprises a first orally acceptable vehicle; a whitening agent; and a fluoride salt that provides fluoride ion in an amount of at least about 1,200 ppm; and the second component comprises a second orally acceptable vehicle and a compound that is incompatible the whitening agent; and

19. The method of claim 20 wherein the applicator is a toothbrush and agitation is effected by brushing the dental surface with the toothbrush.

20. The method of claim 20 wherein the second component of the composition comprises a clay-based thickening agent and an abrasive selected from a siliceous abrasive and an aluminous abrasive.
